(19) United States
(12) Patent Application Publication
(10) Pub. No.: US 2025/0143547 A1
(43) Pub. Date: May 8, 2025

(54) DEVICES, SYSTEMS, AND METHODS FOR SIPHON PREVENTION IN SYSTEMS TO SUPPLY FLUIDS TO AN ENDOSCOPE

(71) Applicant: Boston Scientific Scimed, Inc., Maple Grove, MN (US)

(72) Inventors: Ryan V. Wales, Northborough, MA (US); Scott Edward Corbeil, Litchfield, NH (US); Ryan Vincent William Pollock, Sterling, MA (US); Aakash Deora, Marlborough, MA (US)

(73) Assignee: Boston Scientific Scimed, Inc., Maple Grove, MN (US)

(21) Appl. No.: 18/931,382

(22) Filed: Oct. 30, 2024

Related U.S. Application Data
(60) Provisional application No. 63/595,531, filed on Nov. 2, 2023.

(51) Int. Cl. A61B 1/00; A61B 1/12

(52) U.S. Cl. CPC A61B 1/00131; A61B 1/126

(57) ABSTRACT

Methods and systems for providing a flow of fluid to an endoscope. An illustrative container and tube set arranged and configured to couple to an endoscope may comprise a container configured to contain a fluid, a water supply tube including a first end, a second end, and a first lumen extending therethrough, a gas supply tube including a first end, a second end, and a second lumen extending therethrough, and a fluid flow control assembly positioned in-line with the water supply tube. The fluid flow control assembly may be configured to selectively control a flow of fluid through the water supply tube. The fluid flow control assembly may be configured to allow a flow of fluid in response to an opening pressure.

### CROSS-REFERENCE TO RELATED APPLICATIONS

This application claims the benefit of U.S. Provisional Patent Application Ser. No. 63/595,531 filed on Nov. 2, 2023, the disclosure of which is incorporated herein by reference.

### FIELD

This disclosure relates generally to medical fluid containers and methods, and particularly to a container and tube sets to supply fluid and/or gas to an endoscope.

### BACKGROUND

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. For example, sterile water may be used to irrigate the working lumen during the procedure. Further, during endoscopic procedures, the video lens at the distal end of the endoscope, which is used to navigate and visualize target tissues, may be prone to becoming fouled with blood, mucous, and other debris during the procedure. To generate lens wash, a connector is coupled to an endoscope umbilical via a tube set. The tube set may translate air from the endoscope umbilical to the water container or pressure vessel. A water pickup tube within the container or pressure vessel is fluid contact with the water and coupled to the connector. This allows for the pressure building in the water bottle or pressure vessel to translate the water up the water tube to the distal tip of the endoscope to clean the endoscope lens. The tube set used for providing irrigation fluid and/or lens wash fluid may be commonly used for a period of 24 hours across multiple endoscopic procedures. However, the same endoscope is not used for multiple patients and must be switched out between procedures. When the procedure is over, the connector is disconnected from the umbilical. The water remaining within the tube set may spill on the floor upon disconnection. Additionally, the residual pressure within the connector or pressure vessel may cause a siphon vacuum to be pulled, thus spilling all of the water in the container/pressure vessel out onto the floor via the tube set.

Having a means of preventing water leaking and/or siphoning can prevent the user from needing to clean water on the floor, prevent damage to capital equipment adjacent or under the tubing connector, or requiring the user to get an additional water container or refill the water container for the next procedure. It may be desirable to prevent siphoning and/or leaks of water from the water bottle and/or tube set after the procedure. It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first embodiment, a container and tube set arranged and configured to couple to an endoscope for use in an endoscopic procedure may comprise a container configured to contain a fluid, a water supply tube including a first end, a second end, and a first lumen extending therethrough, wherein the first lumen is in selective fluid communication with the container and the second end of the water supply tube is positioned external to the container, a gas supply tube including a first end, a second end, and a second lumen extending therethrough, wherein the second lumen is in operative fluid communication with the first container and the second end of the first gas supply tube is positioned external to the first container and a fluid flow control assembly positioned in-line with the water supply tube, the fluid flow control assembly configured to selectively control a flow of fluid through the water supply tube. The fluid flow control assembly may be configured to allow a flow of fluid in response to an opening pressure.

Alternatively or additionally to any of the examples above, in another example, the opening pressure may be greater than a head pressure of the fluid within the container.

Alternatively or additionally to any of the examples above, in another example, the fluid flow control assembly may be coupled to the first end of the water supply tube.

Alternatively or additionally to any of the examples above, in another example, the fluid flow control assembly may be coupled adjacent to the second end of the water supply tube.

Alternatively or additionally to any of the examples above, in another example, the fluid flow control assembly may be positioned between the first end and the second end of the water supply tube.

Alternatively or additionally to any of the examples above, in another example, the fluid flow control assembly may be disposed over the water supply tube.

Alternatively or additionally to any of the examples above, in another example, the fluid flow control assembly may comprise a first body member, a valve housing, a lumen extending through the first body member and the valve housing, and a valve extending across the lumen and disposed between the body portion and the valve housing.

Alternatively or additionally to any of the examples above, in another example, the valve may comprise one or more openings extending therethrough.

Alternatively or additionally to any of the examples above, in another example, the valve may comprise a flexible slit valve.

Alternatively or additionally to any of the examples above, in another example, the fluid flow control assembly may further comprise a second body member.

Alternatively or additionally to any of the examples above, in another example, the valve housing may be disposed between the first body member and the second body member.

Alternatively or additionally to any of the examples above, in another example, a first end of the fluid flow control assembly may be coupled to a first segment of the water supply tube and a first segment of the gas supply tube and a second end of the fluid flow control assembly may be coupled to a second segment of the water supply tube and a second segment of the gas supply tube.

Alternatively or additionally to any of the examples above, in another example, the fluid flow control assembly may comprise a valve housing defining a cavity and one or more biasing mechanisms disposed within the cavity. The one or more biasing mechanisms may be configured to exert a biasing force on a wall of the water supply tube to move the wall radially inward.

Alternatively or additionally to any of the examples above, in another example, the valve housing may comprise a flexible inner membrane. The flexible inner membrane may be positioned against the water supply tube.

Alternatively or additionally to any of the examples above, in another example, the one or more biasing mechanisms may comprise a spring.

In another example, a container and tube set arranged and configured to couple to an endoscope for use in an endoscopic procedure may comprise a container configured to contain a fluid, a water supply tube including a first end, a second end, and a first lumen extending therethrough, wherein the first lumen is in selective fluid communication with the container and the second end of the water supply tube is positioned external to the container, a gas supply tube including a first end, a second end, and a second lumen extending therethrough, wherein the second lumen is in operative fluid communication with the first container and the second end of the first gas supply tube is positioned external to the first container, and a fluid flow control assembly positioned in-line with the water supply tube. The fluid flow control assembly may comprise a body member having a first end region having a first outer dimension and a second end region having a second outer dimension, the second outer dimension less than the first outer dimension, a valve housing secured to the second end region of the body member, a lumen extending through the first body member and the valve housing, and a valve extending across the lumen and disposed between the body portion and the valve housing. The fluid flow control assembly may be configured to allow a flow of fluid in response to an opening pressure.

Alternatively or additionally to any of the examples above, in another example, the valve may comprise one or more openings extending therethrough.

Alternatively or additionally to any of the examples above, in another example, the one or more openings may comprise a single slit, one or more slits in the shape of a cross or “x”, a snowflake slit, or a resealable hole.

In another example, a container and tube set arranged and configured to couple to an endoscope for use in an endoscopic procedure may comprise a container configured to contain a fluid, a water supply tube including a first end, a second end, and a first lumen extending therethrough, wherein the first lumen is in selective fluid communication with the container and the second end of the water supply tube is positioned external to the container, a gas supply tube including a first end, a second end, and a second lumen extending therethrough, wherein the second lumen is in operative fluid communication with the first container and the second end of the first gas supply tube is positioned external to the first container, and a fluid flow control assembly positioned in-line with the water supply tube. The fluid flow control assembly may comprise a first body member, a second body member, a valve housing secured between the first body member and the second body member, a third lumen extending from a first end to a second end of the fluid flow control assembly, the third lumen in selective fluid communication with the first lumen of the water supply tube, a fourth lumen extending from the first end to the second end of the fluid flow control assembly, the fourth lumen in fluid communication with the second lumen of the gas supply tube, and a valve extending across the third lumen and disposed between the first body portion and the valve housing. The fluid flow control assembly may be configured to allow a flow of fluid in response to an opening pressure.

Alternatively or additionally to any of the examples above, in another example, the fluid flow control assembly may be configured to be positioned between the first ends and the second ends of the water supply tube and the gas supply tube.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3 depicts another illustrative endoscope system having an alternative fluid supply system;

FIG. 4A is a perspective view of an illustrative flow control assembly;

FIG. 4B is a cross-sectional view of the flow control assembly of FIG. 4A, taken at line 4B-4B of FIG. 4A;

FIG. 5 is a cross-sectional view of the flow control assembly of FIG. 4 coupled to an illustrative connector for connecting an endoscope end of the gas/lens wash supply tubing to the gas/lens wash connector on the connector portion;

FIG. 6A is a perspective view of another illustrative flow control assembly;

FIG. 6B is a cross-sectional view of the flow control assembly of FIG. 6A, taken at line 6B-6B of FIG. 6A;

FIG. 7A depicts a schematic side view of an illustrative fluid reservoir having another illustrative flow control assembly in a first or open configuration; and

FIG. 7B depicts schematic side view of an illustrative fluid reservoir having the flow control assembly of FIG. 7A in a second or closed configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. For example, sterile water may be used to irrigate the working lumen during the procedure. Further, during endoscopic procedures, the video lens at the distal end of the endoscope, which is used to navigate and visualize target tissues, may be prone to becoming fouled with blood, mucous, and other debris during the procedure. To generate lens wash, a connector is coupled to an endoscope umbilical via a tube set. The tube set may translate air from the endoscope umbilical to the water container or pressure vessel. A water pickup tube within the container or pressure vessel is fluid contact with the water and coupled to the connector. This allows for the pressure building in the water bottle or pressure vessel to translate the water up the water tube to the distal tip of the endoscope to clean the endoscope lens. The tube set used for providing irrigation fluid and/or lens wash fluid may be commonly used for a period of 24 hours across multiple endoscopic procedures. However, the same endoscope is not used for multiple patients and must be switched out between procedures. When the procedure is over, the connector is disconnected from the umbilical. The water remaining within the tube set may spill on the floor upon disconnection. Additionally, the residual pressure within the connector or pressure vessel may cause a siphon vacuum to be pulled, thus spilling all of the water in the container/pressure vessel out onto the floor via the tube set. Having a means of preventing water leaking and/or siphoning can prevent the user from needing to clean water on the floor, prevent damage to capital equipment adjacent or under the tubing connector, or requiring the user to get an additional water container or refill the water container for the next procedure. Disclosed herein are devices and systems to prevent siphoning and/or leaks of water from the water bottle and/or tube set after the procedure

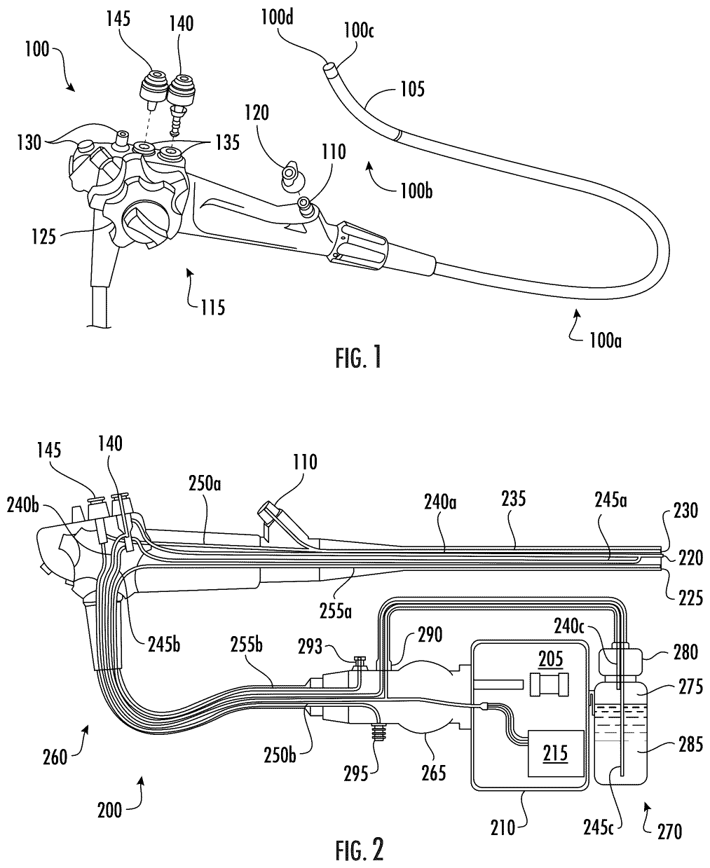

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top of the water reservoir. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

It is contemplated that other arrangements for the fluid sources may be used as desired. For example, in some cases, water for irrigation and lens wash may come from a same container. Some illustrative systems and method to supply fluids to the endoscope are described in commonly assigned U.S. Patent Application No. 63/419,900, titled DEVICES, SYSTEMS, AND METHODS TO SUPPLY FLUIDS TO AN ENDOSCOPE, the disclosure of which is hereby incorporated by reference.

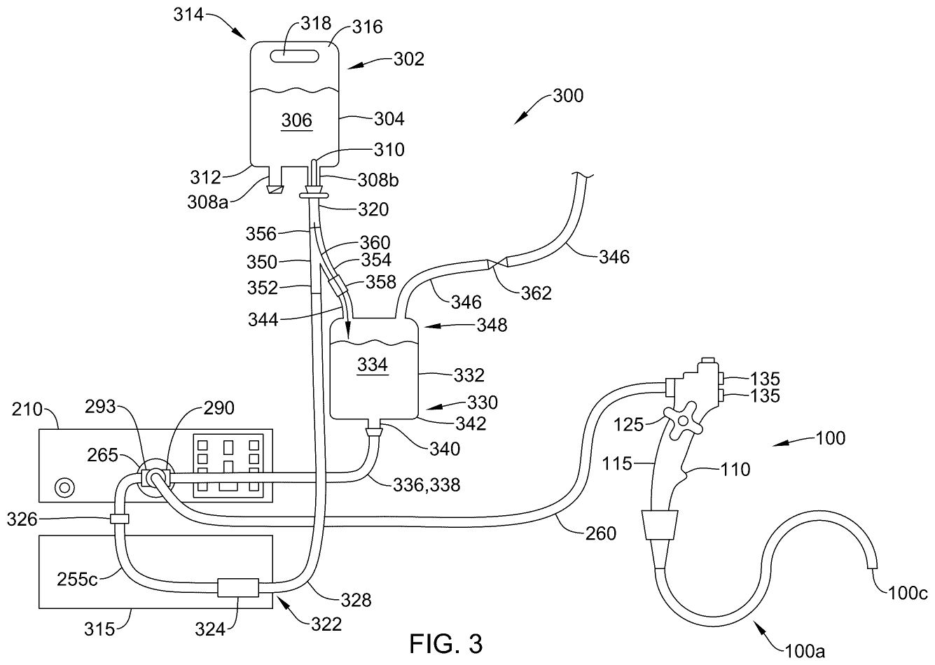

FIG. 3 depicts a schematic view of another illustrative endoscopic system 300 which may reduce the number of water reservoir changes and/or reduce opportunities for contamination during replacement of the water reservoir(s). The system 300 may include a number of advantages over the current bottle system described above. The system 300 may include components similar to the endoscope and endoscope systems described with regard to FIGS. 1-2; however, not all features may be described or shown here.

Generally, the system 300 may include a first reservoir 302 and a second reservoir 330. The first reservoir 302 may be configured to supply water or fluid for both irrigation (e.g., via the first reservoir 302) and lens wash (e.g., via the second reservoir 330). This may allow a single fluid source to be used to provide fluid for both irrigation and lens wash. While not explicitly shown, the reservoirs 302, 330 may include printed lines, numbers, or other visual indicia to allow a user to easily determine how much fluid is left in the reservoirs 302, 330.

The first reservoir 302 may include a first container 304 configured to hold a first volume of fluid 306. In the illustrated embodiment, the first container 304 is fluidly coupled to the upstream irrigation supply tubing 328 and is configured to provide fluid for irrigation to the endoscope 100. Generally, the irrigation supply tubing 328 may be a water or fluid supply line or tube for supplying water or other fluid to an endoscope. Additionally, the first container 304 may be selectively fluidly coupled to a second fluid reservoir 330. The second reservoir 330 may include a second container 332 configured to hold a second volume of fluid 334. In the illustrated embodiment, the second container 332 is fluidly coupled to the gas and lens wash supply tubing 336, 338 and is configured to provide fluid for lens wash to the endoscope 100. Generally, the lens wash supply tubing 338 may be a water or fluid supply line or tube for supplying water or other fluid to an endoscope. The gas and lens wash supply tubing 336, 338 may be coaxially arranged. For example, the gas supply tubing 336 may define a lumen that is sufficiently large in diameter to encompass a smaller diameter lens wash tubing 338, coaxially received within the gas supply tubing 336, as well as provide air to the water source in an annular space surrounding the lens wash tubing 338 to pressurize the second reservoir 330. The lens wash supply tubing 338 may be configured to exit the lumen defined by the coaxial gas supply tubing 336 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 336, 338 may be arranged in a side-by-side arrangement.

The first and second containers 304, 332 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 304, 332 may be entirely translucent, entirely opaque, or combinations thereof. In some cases, the first and second containers 304, 332 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 304, 332 may be variable. For example, the volume of the first container 304 and/or the second container 332 may be 500 milliliters (mL) or greater, 1000 mL or greater, 2000 mL or greater, 3000 mL, 4000 mL or greater, etc. The volume may be less than 500 mL or greater than 4000 mL, as desired. One or both of the first and second reservoirs 302, 330 may be pre-filled (e.g., prior to entering the procedure suite or at the time of manufacturing) with water or other fluid. In some cases, the clinician may select the reservoir(s) 302, 330 from a plurality of differently sized available reservoirs based on the number and/or types of procedures expected for a typical day or the specific day. In the illustrated embodiments, the first reservoir 302 may supply fluid to the second reservoir 330. By selecting a first reservoir 302 having a volume large enough to accommodate an entire day of procedures, the need for replacing the sterile fluid source (e.g., the first reservoir 302) may be reduced or eliminated. In some cases, the first reservoir 302 may be used to periodically refill the second reservoir 330. Thus, the volume of the first reservoir 302 may be greater than the volume of the second reservoir 330, although this is not required. It is further contemplated that, in some embodiments, one or both of the first or second reservoirs 302, 330 may be a rigid bottle.

It is contemplated that flexible bags may utilize less plastic (or other material) than a bottle designed to hold a similar amount of fluid. Thus, the use of a flexible bag as a fluid reservoir 302, 330 may increase the level of environmental sustainability of the system 300. For example, if the user sets up the system with a 3000 mL (3 liter) bag reservoir 302 and therefore does not need to utilize three individual one liter bottles, a significant reduction of waste may be realized. It is further contemplated that when disposed of or discarded, a flexible bag reservoir may occupy less volume than a bottle capable of holding an equivalent amount of fluid.

The first reservoir 302 may further include one or more ports 308a, 308b, such as, but not limited to a spike port or a septum port, extending from and in selective fluid communication with an interior of the first container 304. The ports 308a, 308b may be formed as a monolithic structure with the first container 304. The ports 308a, 308b may be generally tubular structures with each port 308a, 308b defining a lumen extending therethrough. The lumens of the ports 308a, 308b may be configured to selectively fluidly couple the interior of the first container 304 with another component, such as, but not limited to, a fluid or water supply tube. In some embodiments, the ports 308a, 308b may be positioned adjacent to a bottom end 312 of the first reservoir 302. However, this is not required. The ports 308a, 308b may be positioned in other locations, as desired. If the ports 308a, 308b are positioned at a location other than the bottom end 312 of the first container 304, a dip tube or tube extension may be required to access the fluid at the bottom of the first container 304. In some cases, at least one port 308b may be configured to be coupled to the upstream irrigation tubing (or water supply tube) 328 while another port 308a may be configured to allow the user to add additives to the fluid 306. In other examples, the upstream irrigation tubing 328 may be coupled to the first port 308a while the second reservoir 330 is in fluid communication with the second port 308b. While the first reservoir 302 is illustrated as including two ports 308a, 308b, the first reservoir 302 may include one port or more than two ports, as desired.

While not explicitly shown, the ports 308a, 308b may each include a removable cap or seal configured to form a fluid tight seal with the port 308a, 308b. The removable cap or seal may help to maintain the sterility of the ports 308a, 308b. The removable cap or seal may be coupled to a free end of the ports 308a, 308b using a number of different techniques. For example, the cap or seal may be coupled to the port 308a, 308b using a threaded engagement, a friction fit, a snap fit, etc. In other instances, the cap or seal may be removed through a twisting motion configured to break the cap or seal from the port 308a, 308b. Once the cap or seal has been removed, the port 308a, 308b may be pierced with a spike tip or spike port adaptor 310 that is coupled to the upstream irrigation tubing 328. For example, in addition to the removable cap or seal, the port 308a, 308b may include an internal seal disposed within a lumen of the port 308a, 308b that may be punctured or pierced by the spike port adaptor 310. The internal seal may be configured to prevent fluid 306 from leaking from the first container 304 prior to the spike port adaptor 310 being inserted into the port 308a, 308b. In some embodiments, the internal seal may be self-sealing such that upon removal of the spike port adaptor 310 fluid is prevented from leaking from the port 308a, 308b. The outer surface of the spike port adaptor 310 may form an interference fit with the inner surface of the port 308a, 308b. The fit and/or coupling between the spike port adaptor 310 and the port 308a, 308b may be sufficient to remain in place when the irrigation supply tube 328, branched connector 350, and/or other tubing sets are coupled to the spike port adaptor 310. It is contemplated that the spike port adaptor 310 may be inserted into one of the ports 308a, 308b utilizing universally used aseptic techniques such as those used with IV fluid bags. This may help reduce infection risk by maintaining sterile components, not introducing contaminants into the fluid 306, etc. It is further contemplated that additives may be added to the fluid 306 using similar aseptic techniques via one of the ports 308a, 308b.

The first reservoir 302 may include a handle 316 positioned adjacent to a top portion 314 thereof. The handle 316 may define an opening or through hole 318 for receiving a hand or hook therethrough to carry the first reservoir 302. In some cases, the handle 316 may include an undulating surface configured to provide a more ergonomic grip for the user. It is contemplated that the handle 316 may be formed from a similar material as the first container 304 or a different material, as desired. In some examples, the handle 316 may be formed from polyethylene terephthalate (PET), polypropylene (PP), etc. The handle 316 may allow the first reservoir 302 to be hung from a hook, such as, but not limited to an IV stand. Hanging the first reservoir 302 may allow the first reservoir 302 to be positioned above the level of an endoscope cart which may enable the user to see the fluid 306 level at any time. This may help the clinician avoid running out of fluid during a procedure. Additionally, elevating the reservoir may eliminate the need for the clinician to bend or stoop during setup of the system 300 and/or to change the first reservoir 302. In some cases, head pressure generated from the elevating the first reservoir 302 may enable rapid priming of the irrigation circuit (and/or lens wash circuit if so connected) which may save time during setup. It is further contemplated that hanging the first reservoir 302 from a hook or IV stand may allow the first reservoir 302 to be positioned away from expensive capital equipment thus reducing or eliminating the potential for fluid running or flowing inadvertently onto the capital equipment and causing damage or destruction.

The first reservoir 302 may be connected in fluid communication with a lumen of the upstream irrigation supply tube 328. The upstream irrigation supply tube 328 extends from a second end region 322 external to the container 304 and positioned within a pump head 324 of the peristaltic irrigation pump 315 to a first end 320. The first end 320 of the upstream irrigation supply tube 328 is coupled to the spike port adaptor 310 which in turn is configured to extend through a lumen of the port 308b and pierce a seal within the lumen of the port 308b to fluidly couple the interior of the container 304 with the lumen of the upstream irrigation supply tube 328. The second end of the upstream irrigation supply tube 328 is configured to be fluidly coupled with an irrigation lumen of the endoscope 100. When irrigation water is required, fluid is pumped from the first container 304 by operating the irrigation pump 315, such as by depressing a footswitch (not shown), and flows from the first reservoir 302, through the upstream irrigation supply tubing 328 and a branched connector 350, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in line with the downstream irrigation supply tubing 255c. The flow control valve 326 may prevent the unintentional flow of fluid from the first container 304 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 302 and the irrigation pump 315. The flow control valve 326 may also prevent fluid from leaking from the downstream irrigation supply tube 255c when the endoscope 100 is changed between patients and the tubing set connector is separated from the endoscope water port.

In some embodiments, the irrigation pump 315 may be omitted. For example, the reservoir 302 may be inserted into a compression sleeve. When irrigation fluid is desired, the compression sleeve may be activated to exert pressure on an outer surface of the reservoir 302 and to provide the required pressure to perform irrigation at the distal end of the endoscope 100. In another embodiment, the reservoir 302 may be inserted into a compression sleeve which applies constant pressure to the reservoir 302 with a flow switch positioned along irrigation supply tubing 328 to provide binary control of irrigation flow.

The second reservoir 330 may further include one or more ports 340, such as, but not limited to a spike port or a septum port, extending from and in selective fluid communication with an interior of the second container 332. The port 340 may be formed as a monolithic structure with the second container 332. The port 340 may be a generally tubular structure with the port 340 defining a lumen extending therethrough. The lumen of the port 340 may be configured to selectively fluidly couple the interior of the second container 332 with another component, such as, but not limited to, fluid/water/gas supply tube(s). In some cases, the port 340 may be configured to be coupled to the gas and lens wash supply tubing 336, 338. In some embodiments, the port 340 may be positioned adjacent to a bottom end 342 of the second reservoir 330. However, this is not required. The port 340 may be positioned in other locations, as desired. If the port 340 is positioned at a location other than the bottom end 342 of the second container 332, a dip tube or tube extension may be required (e.g., coupled to the lens wash supply tubing 338) to access the fluid at the bottom of the second container 332. While the second reservoir 330 is illustrated as including one port 340, the second reservoir 330 may include more than one port, as desired.

While not explicitly shown, the port 340 may include a removable cap or seal configured to form a fluid tight seal with the port 340. The removable cap or seal may help to maintain the sterility of the port 340. The removable cap or seal may be coupled to a free end of the port 340 using a number of different techniques. For example, the cap or seal may be coupled to the port 340 using a threaded engagement, a friction fit, a snap fit, etc., or may be fixedly coupled using a number of techniques such as adhesive or solvent bonding. In other instances, the cap or seal may be removed through a twisting motion configured to break the cap or seal from the port 340. Once the cap or seal has been removed, the port 340 may be pierced with a spike tip or spike port adaptor (not explicitly shown) that is coupled to the gas and lens wash supply tubing 336, 338. For example, in addition to the removable cap or seal, the port 340 may include an internal seal disposed within a lumen of the port 340 that may be punctured or pierced by the spike port adaptor. The internal seal may be configured to prevent fluid 334 from leaking from the second container 332 prior to the spike port adaptor being inserted into the port 340. In some embodiments, the internal seal may be self-sealing such that upon removal of the spike port adaptor fluid is prevented from leaking from the port 340. The outer surface of the spike port adaptor may form an interference fit with the inner surface of the port 340. The fit and/or coupling between the spike port adaptor and the port 340 may be sufficient to remain in place when the gas and fluid supply tubing 336, 338 and/or other tubing sets are coupled to the spike port adaptor. It is contemplated that the spike port adaptor may be inserted into the port 340 utilizing universally used aseptic techniques such as those used with IV fluid bags. This may help reduce infection risk by maintaining sterile components, not introducing contaminants into the fluid 334, etc. It is further contemplated that additives may be added to the fluid 334 using similar aseptic techniques via the port 340, if so desired. In some cases, other coupling mechanisms may be used as desired to couple the gas and lens wash supply tubing 336, 338 to the port 340. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 336 extends from a second end external to the second container 332 to the port 340. The gas supply tubing 336 may extend into the interior of the second container 332 and terminate within a reservoir gap (e.g., above the level of the fluid 334). However, in some cases, the gas supply tubing 336 may terminate within the fluid 334. A lumen extends through the gas supply tubing 336 for receiving a flow of air and/or gas therethrough. The lumen of the gas supply tubing 336 may be in operative fluid communication with a top portion of the interior of the second container 332. The lens wash supply tubing 338 extends from a second end external to the second reservoir 330 to a first end in fluid communication with a bottom portion 342 of the second container 332. In some embodiments, the lens wash supply tubing 338 may terminate at the port 340. A lumen extends through the lens wash supply tubing 338 for receiving a flow of fluid therethrough. The lumen of the lens wash supply 338 is in selective operative fluid communication with a bottom portion 342 of the second container 332. In the illustrated embodiment, the gas supply tubing 336 and the lens wash supply tubing 338 may couple to the second container 332 through a single or common opening (e.g., port 340). For example, the gas supply tubing 336 and the lens wash supply tubing 338 may be coaxially arranged. However, this is not required. In some cases, the gas supply tubing 336 and the lens wash supply tubing 338 may extend in a side by side arrangement or may be separately connected to the second container 332 in different locations.

The second container 332 may further include a first fluid inlet 344 and a second fluid inlet 346. While the first and second fluid inlets 344, 346 are illustrated as being adjacent to or extending from a top portion 348 of the second container 332, the first and/or second fluid inlets 344, 346 may be positioned at other locations about the second container 332, as desired. In some embodiments, the first and/or second fluid inlets 344, 346 may be tubular members formed as a single monolithic structure with the second container 332. In other embodiments, the first and/or second fluid inlets 344, 346 may include tubular components releasably coupled to ports (similar in form and function to port 340) formed in or with the container 332.

The first fluid inlet 344 may be in selective fluid communication with the first reservoir 302. For example, a branched connector 350 may be positioned in-line with the upstream irrigation tubing 328. In some embodiments, the branched connector 350 may be a “Y” connector or a “T” connector having an inlet leg 356 defining a first fluid inlet, a first outlet leg 352 defining a first fluid outlet, and a second outlet leg 354 defining a second fluid outlet. However, it is contemplated that the branched connector 350 may include more than one fluid inlet and fewer than two or more than two fluid outlets, if so desired.

The branched connector 350 may be positioned in-line with the upstream irrigation tubing 328 such that the inlet leg 356 and the first outlet leg 352 are fluidly coupled with the lumen of the upstream irrigation tubing 328. Fluid may flow from the first reservoir 302, through the upstream irrigation tubing 328, through the branched connector 350 and again through the upstream irrigation tubing 328. The branched connector 350 may be positioned such that the inlet leg 356 is upstream of the outlet legs 352, 354 relative to a flow of irrigation fluid. In some embodiments, the branched connector 350 and the spike port 310 may be molded or formed as a single monolithic structure. It is contemplated that this may reduce connection points in the fluid circuit. In such an instance, the first end 320 of the irrigation supply tubing 328 may be fluidly coupled to the first outlet leg 352 of the branched connector 350.

The second outlet leg 354 may be fluidly coupled to the first fluid inlet 344 of the second reservoir 330. A flow control mechanism, such as, but not limited to, a one-way valve 358 may be positioned between the second fluid outlet of the second outlet leg 354 and the first fluid inlet 344 of the second reservoir 330 to selectively fluidly couple the second container 332 with the first container 304. The one-way valve 358 may be configured to be opened to allow fluid to selectively pass from the first reservoir 302 to the second reservoir 330 while preventing fluid (e.g., gas, water, or other fluid) from exiting the second container 332 and entering the irrigation supply tubing 328 and/or the first container 304. In some embodiments, the one-way valve 358 may be replaced with a clamp which may compress the first fluid inlet 344 to selectively fluidly isolate the second container 332 from the first container 304 and removed to selectively couple the second container 332 with the first container 304. In yet other embodiments, the one-way valve 358 may be replaced with a spring-loaded valve, a stopcock, or other two-way valve. When it is desired to add fluid to the second reservoir 330 from the first reservoir 302, the one-way valve 358 (or other flow control mechanism) may be opened or released. Fluid may then be at least partially diverted from the irrigation supply tubing 328 through the second outlet leg 354 of the branched connector 350 and into the second container 332 along flow path 360. Fluid may be added to the second container 332 while the irrigation pump 315 is running or while the irrigation pump 315 is idle, as desired.

The second fluid inlet (or gas supply tube) 346 of the second container 332 may be an alternative gas supply tubing configured to be coupled to an alternative gas supply (e.g., CO₂ hospital house gas source). The second fluid inlet 346 may extend from a second end external to the second container 332 to a first end coupled to the second container 332. The alternative gas supply may be used to pressurize the second container 332 to supply lens wash to the endoscope 100 and/or to provide insufflation. A lumen extends through the second fluid inlet 346 for receiving a flow of gas therethrough. The lumen of the second fluid inlet 346 is in operative fluid communication with a top portion of the second container 332. The flow of the CO₂ through the system 300 may be similar to that described above. For example, in the neutral state, CO₂ gas flows through the second fluid inlet 346 into the second container 332, up the gas supply tubing 336 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid inlet 346 into the second container 332, up the gas supply tubing 336 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the second reservoir 330 is maintained by delivering gas through the second fluid inlet 346. It is contemplated that the one-way valve 358 is in the closed configuration during delivery of the CO₂ gas to allow the container 332 to pressurize. In some instances, the one-way valve 358 may be configured to close without user intervention in response to the delivery of CO₂ to the second container 332. In some embodiments, the system 300 may include a branched connector (such as, but not limited to a “Y” or “T” connector) at the second fluid inlet 346 to allow either air or CO₂ to be used for pressurization or insufflation. It is further contemplated that the second fluid inlet 346 may include a pressure relief valve 362, such as, but not limited to, a 3-way stopcock, a clamp, or a spring-loaded valve, to vent pressure within the second container 332 and/or to block a flow of pressurized gas to the second container 332 during refilling of the second container 332, during procedure change-overs, and/or during equipment change-overs.

It is contemplated that the use of a flexible bag in place of a rigid bottle for the second reservoir 330 may reduce or eliminate the risk of air leaking from bottle and cap connections. This may eliminate the need for clinicians to attempt to remedy the leak by adjusting the cap and bottle assemblies or from discarding a cap and/or bottle if the leak cannot be remedied.

As the pressurized second container 332 is fluidly isolated from the first container 304 when the one-way valve 358 is closed, it is contemplated that the clinician may replace the first reservoir 302 with a new (full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 302 with a new full bag, for example when the first reservoir 302 is empty or near empty, the user may hang the new bag near the first reservoir 302 to be replaced. The user may then disengage the spike port adaptor 310 from the port 308b and insert the spike port adaptor 310 into a port of the new bag. This may be performed without requiring the clinician to bend or stoop to access the first reservoir 302. The port 308b may self-seal to prevent fluid leaks from the first reservoir 302 being replaced. This method of replacing the first reservoir 302 may have a lower risk of introducing contaminants into the systems relative to traditional bottle systems. For example, the change out method described herein may allow the first reservoir 302 to be changed out without having tubing dangling from a cap (as in a bottle system). Further, the system 300 may remain largely closed as the first reservoir 302 is changed out.

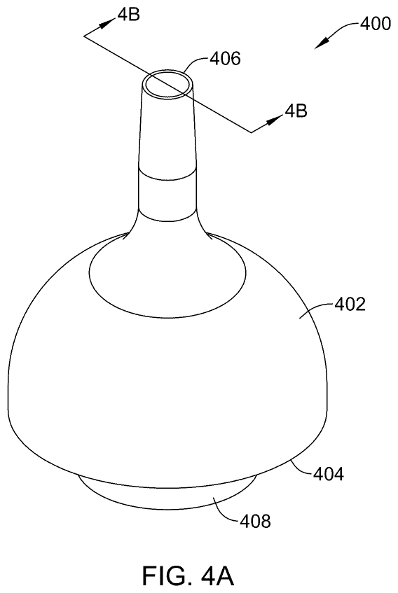

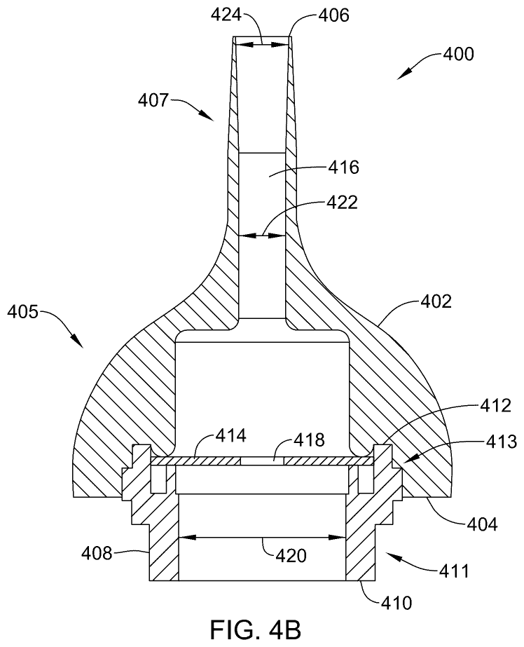

As described above, it may be desirable to prevent water/fluid from leaking out of the gas/lens wash supply tubing 240c, 245c, 336, 338 (e.g., configured to be connected to gas/lens wash connector 290) and/or the irrigation tubing 255c, 328 (e.g., configured to be connected to the irrigation connector 293). It is contemplated that the flow of fluid may be selectively stopped or prevented at a number of different locations in the fluid circuit. FIG. 4A is a perspective view of an illustrative fluid flow control assembly 400. FIG. 4B is a cross-sectional view of the fluid flow control assembly 400 of FIG. 4A, taken at line 4B-4B of FIG. 4A. The fluid flow control assembly 400 may be configured to be coupled to a first end of the lens wash supply tubing 245c, 338 to selectively allow a flow of fluid through the lens wash supply tubing 245c, 338. The fluid flow control assembly 400 may be disposed within an interior of the reservoir 270, 330. In some cases, in addition to selectively controlling a flow of fluid, the fluid flow control assembly 400 may also maintain a desired position of the first end of the lens wash supply tubing 245c, 338 within the reservoir 270, 330. For example, when the lens wash supply tubing 245c, 338 extends through a lateral side or a top of the reservoir 270, 330, the lens wash supply tubing 245c, 338 may extend to a bottom of the reservoir 270, 330 to access fluid at the bottom of the reservoir 270, 330. The fluid flow control assembly 400 may be configured to maintain the first end of the lens wash supply tubing 245c, 338 adjacent to the bottom of the reservoir 270, 330. In some cases, a weight may be coupled to the first end of the lens wash supply tubing 245c, 338 to prevent the lens wash supply tubing 245c, 338 from floating to a top of the fluid within the reservoir 270, 330. Thus, the fluid flow control assembly 400 may have a sufficient density (e.g., greater than the density of water or the fluid within the reservoir 270, 330) to maintain the first end of the lens wash supply tubing 245c, 338 in fluid communication with a bottom portion of the reservoir 270, 330.

The fluid flow control assembly 400 may include a body member 402 extending from a first end 404 to a second end 406. The first end 404 of the body member 402 may be configured to receive a valve housing 408 while the second end 406 of the body member 402 may be configured to be coupled to the first end of the lens wash supply tubing 245c, 338. In some examples, the first end 404 of the body member 402 may have a first cross-sectional outer dimension and the second end 406 of the body member 402 may have a second cross-sectional outer dimension smaller than the first cross-sectional outer dimension. The first end region 405 of the body member 402 may take any shape desired such, as, but not limited to, generally hemi-spherical, conical, cylindrical, rectangular prisms, and the like. The second end region 407 of the body member 402 may have a generally tubular configuration sized and shaped to couple with the first end of the lens wash supply tubing 245c, 338. In some embodiments, the second end region 407 may be disposed within a lumen of the lens wash supply tubing 245c, 338. In other embodiments, the lens wash supply tubing 245c, 338 may be disposed within a lumen 416 of the fluid flow control assembly 400.

The valve housing 408 may extend from a first end 410 to a second end 412. A second end region 413 of the valve housing 408 may be coupled to the first end region 405 of the body member 402. It is contemplated that the second end region 413 of the valve housing 408 may be coupled to the body member 402 using a variety of techniques, such as, but not limited to, threaded engagements, snap fits, press fits, friction fits, overmolding, adhesive bonding, welding, soldering, and the like. A first end region 411 of the valve housing 408 may extend beyond the first end 404 of the body member 402. However, this is not required. A valve 414 may be positioned between the valve housing 408 and the body member 402 within a lumen 416 extending from the first end to the second of the fluid flow control assembly 400 (e.g., from the first end 410 of the valve housing 408 to the second end 406 of the body member 402). The valve 414 may extend across a cross-section of the lumen 416 and may include one or more openings or passages 418 to selectively allow a flow of fluid through the valve 414.

The lumen 416 may have a first cross-sectional dimension 420 adjacent to the first end region 411 of the valve housing 408 and a second, smaller, cross-sectional dimension 422 adjacent to the second end region 407 of the body member 402. The transition between the first larger cross-sectional dimension 420 and the second smaller cross-sectional dimension 422 may occur between the valve 414 and the second end 406 of the body member 402. The transition in cross-sectional dimensions may be an abrupt stair step transition or a gradual sloped transition, as desired. It is further contemplated that the lumen 416 may have a third cross-sectional dimension 424 adjacent to the second end 406 of the body member 402. The third cross-sectional dimension 424 may be greater than the second cross-sectional dimension 422, although this is not required. In some embodiments, the transition from the second cross-sectional dimension 422 to the third cross-sectional dimension 424 may be a gradual sloped transition. The sloped inner surface of the second end region 407 of the body member 402 may form a friction fit with the lens wash supply tubing 245c, 338.

The valve 414 may be configured to prevent fluid from freely flowing into or out of the lens wash supply tubing 245c, 338 unless a minimum opening pressure is achieved. The opening pressure may be greater than the head pressure of the fluid 285, 334 within the reservoir 270, 330. It is contemplated that the minimum pressure may be achieved by the pressure generated by the flow of air/gas through the gas supply line 240c, 336 and into the interior of the reservoir 270, 330. Once the minimum pressure is achieved, the valve 414 may open and water/fluid may flow through the lens wash supply tubing 245c, 338 at a rate sufficient to clean the lens of the endoscope 100. Once the clinician releases the gas/water valve 140, the reservoir 270, 330 may return to its static pressure which may close the valve 414 and prevent the flow of fluid into and/or out of the lens wash supply tubing 245c, 338. The valve 414 may be formed from a single monolithic piece of silicone, thermoplastic elastomer (TPE), elastic, other flexible material, or the like. In some cases, the valve 414 may be formed from more than one piece. The one or more openings may be a single slit, one or more slits in the shape of a cross or “x”, a snowflake slit, a single resealable hole, or the like. In other embodiments, the valve 414 may be an umbrella valve that changes configuration in response to the pressure change. It is further contemplated that other valves which open in response to an increase in pressure and close as the pressure is lowered may also be used.

The valve 414 may be configured to remain closed under the greatest head pressure by the water/fluid 285, 334 within the reservoir (e.g., when the reservoir 270, 330 is full). Performance characteristics of the valve 414 such as, but not limited to, the head pressure the valve 414 can resist, the flow rate and/or volume of fluid flow through the valve 414 when the opening pressure is reached can be adjusted by changing the size and/or shape of the valve 414 and/or the opening 418, material selection of the valve 414, material properties of the valve 414, the design of the body member 402 and/or valve housing 408.

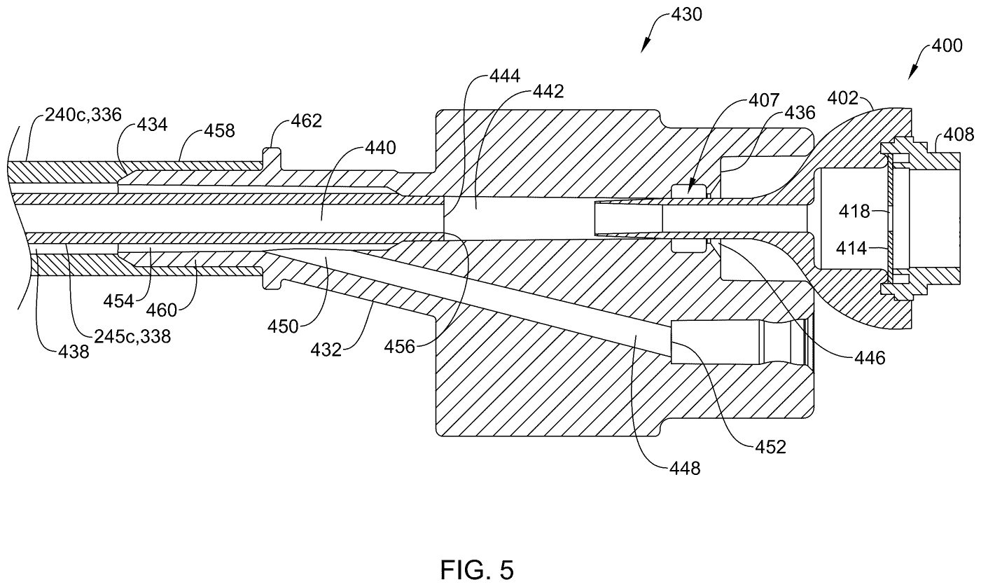

In some embodiments, the fluid flow control assembly 400 may be positioned adjacent to the second end of the lens wash supply tubing 245c, 338. For example, the fluid flow control assembly 400 may be positioned between the second end of the lens wash supply tubing 245c, 338 and the gas/lens wash connector 290. FIG. 5 depicts a cross-sectional view of the fluid flow control assembly 400 coupled to an illustrative connector 430 for connecting an endoscope end of the gas/lens wash supply tubing 240c, 245c, 336, 338 to the gas/lens wash connector 290 on the connector portion 265.

The connector 430 may include a housing 432 extending from a first inlet end 434 to a second outlet end 436. The first inlet end 434 may be configured to be coupled to the gas supply tubing 240c, 336 and the lens wash supply tubing 245c, 338 and the second outlet end 436 may be configured to be coupled to the gas/lens wash connector 290 on the connector portion 265. In some embodiments, the fluid flow control assembly 400 may be positioned between the second outlet end 436 of the connector 430 and the gas/lens wash connector 290. The gas supply tubing 240c, 336 and lens wash supply tubing 245c, 338 may be combined in a coaxial arrangement. For example, the gas supply tubing 240c, 336 may define a lumen 438 that is sufficiently large in diameter to encompass a smaller diameter lens wash supply tubing 245c, 338, coaxially received within the gas supply tubing 240c, 336, as well as provide air to the water source in an annular space surrounding the lens wash supply tubing 245c, 338 to pressurize the water reservoir. The lens wash supply tubing 245c, 338 may be configured to exit the lumen 438 defined by the coaxial gas supply tubing 240c, 336 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion (e.g., connector portion 265 of FIG. 2). In the illustrated embodiment, the housing 432 may transition the lumens 438, 440 of the gas supply tubing 240c, 336 and the lens wash supply tubing 245c, 338 to a side-by-side arrangement. In some embodiments, the gas supply tubing 240c, 336 and the lens was supply tubing 245c may be coupled to the first inlet end 434 of the housing 432 in a side-by-side arrangement.

The housing 432 may define a first fluid lumen 442 extending from a first fluid inlet 444 to a first fluid outlet 446 and a second lumen 448 extending from a second fluid inlet 450 to a second fluid outlet 452. The first and second lumens 442, 448 may extend from or branch from a shared fluid lumen 454. The fluids traveling through the first and second lumens 442, 448 may be fluidly isolated from one another in the shared fluid lumen 454 via the gas/lens wash supply tubing 240c, 245c. For example, the lens wash supply tubing 245c, 338 may extend distally beyond the second fluid inlet 450 to fluidly isolate the first and second lumens 442, 448.

It is contemplated that a location of the first fluid inlet 444 may vary along a length of the housing 432 and may at least partially depend on a location of the second end 456 of the lens wash supply tubing 245c, 338. The first fluid lumen 442 may be in fluid communication with a lumen 440 of the lens wash supply tubing 245c, 338 to supply lens wash fluid to the endoscope. Fluid may exit an opening of the lens wash supply tubing 245c, 338 at the second end 456 thereof and enter the first fluid lumen 442 of the housing 432. In some embodiments, a length of the lens wash supply tubing 245c, 338 may extend coaxially through the shared fluid lumen 454 such that the second end thereof is positioned between the first and second ends 434, 436 of the housing 432. In some embodiments, a diameter of the shared fluid lumen 454 may taper or reduce in diameter towards the second end 436 of the housing 432. An outer surface of the lens wash supply tubing 245c, 338 may frictionally engage an inner surface of the housing 432 defining the shared fluid lumen 454 and/or the first fluid lumen 442 adjacent a reduced diameter portion thereof. This may fluidly isolate the first fluid outlet 446 from the second fluid outlet 452.

A second end 458 of the gas supply tubing 240c, 336 may be disposed over an outer surface of the housing 432 adjacent the first end 434 thereof. For example, the second end 458 of the gas supply tubing 240c, 336 may be disposed over and frictionally engage a neck portion 460 of the housing 432 to provide a gas-tight coupling between the gas supply tubing 240c, 336 and the housing 432. In some embodiments, the housing 432 may include a radially extending protrusion or ridge 462 configured to provide a mechanical stop for the second end 458 of the gas supply tubing 240c, 336. In other embodiments, the second end of the gas supply tubing 240c, 336 may be disposed and secured within the shared fluid lumen 454. The second fluid lumen 448 of the housing 432 may be in fluid communication with the lumen 438 of the gas supply tubing 240c, 336. Air/gas may enter the second fluid lumen 448 via the second fluid inlet 450. The engagement of the outer surface of the lens wash supply tubing 245c, 338 and the inner surface of the housing 432 defining the shared fluid lumen 454 and/or the first fluid lumen 442 may prevent air/gas within the lumen 438 of the gas supply tubing 240c, 336 from entering the first fluid lumen 442 of the housing 432.

The fluid flow control assembly 400 may be coupled with the connector 430 by inserting the second end region 407 of the body member 402 into the first fluid lumen 442. The fluid flow control assembly 400 may selectively fluidly couple the first fluid inlet 444 with the gas/lens wash connector 290. As described herein, the valve 414 may be configured to prevent fluid from freely flowing into or out of the lens wash supply tubing 245c, 338 unless a minimum opening pressure is achieved. The opening pressure may be greater than the head pressure of the fluid 285, 334 within the reservoir 270, 330. It is contemplated that the minimum pressure may be achieved by the pressure generated by the flow of air/gas through the gas supply line 240c, 336 and into the interior of the reservoir 270, 330. Once the minimum pressure is achieved, the valve 414 may open and water/fluid may flow through the lens wash supply tubing 245c, 338 at a rate sufficient to clean the lens of the endoscope 100. Once the clinician releases the gas/water valve 140, the reservoir 270, 330 may return to its static pressure which may close the valve 414 and prevent the flow of fluid into and/or out of the lens wash supply tubing 245c, 338.

The valve 414 may be configured to remain closed under the greatest head pressure by the water/fluid 285, 334 within the reservoir (e.g., when the reservoir 270, 330 is full). Performance characteristics of the valve 414 such as, but not limited to, the head pressure the valve 414 can resist, the flow rate and/or volume of fluid flow through the valve 414 when the opening pressure is reached can be adjusted by changing the size and/or shape of the valve 414 and/or the opening 418, material selection of the valve 414, material properties of the valve 414, the design of the body member 402 and/or valve housing 408.

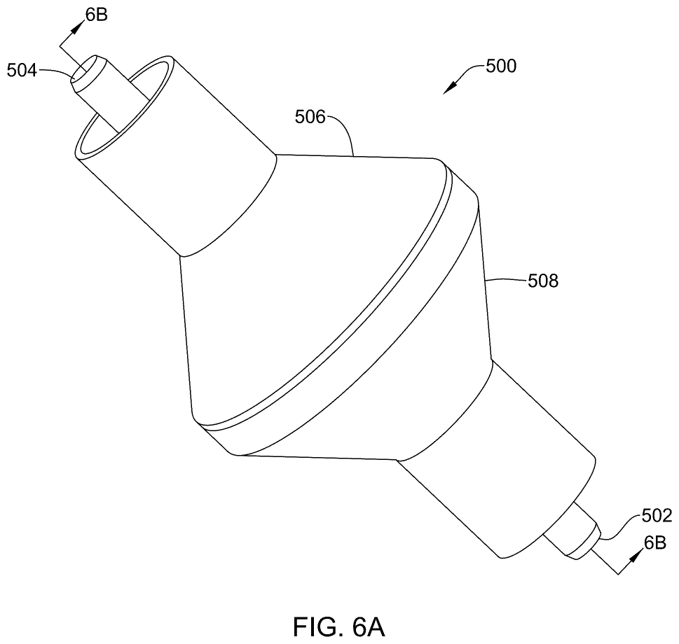

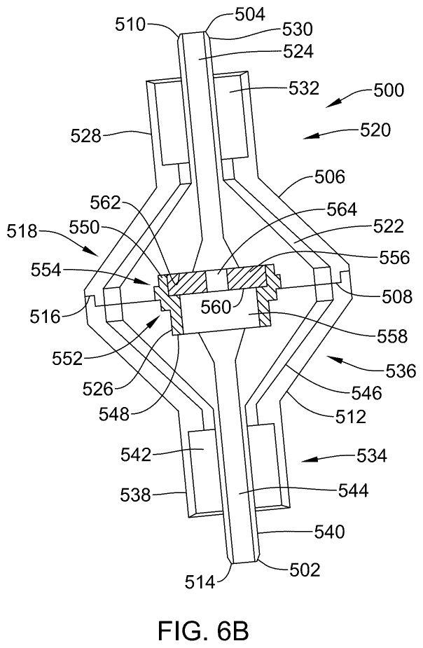

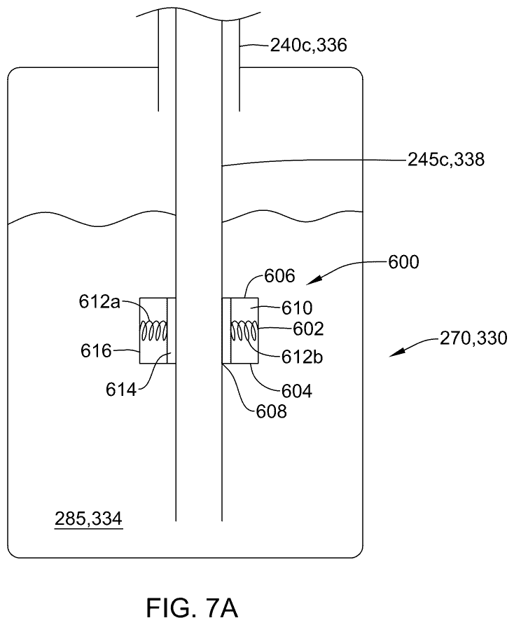

FIG. 6A is a perspective view of another illustrative fluid flow control assembly 500. FIG. 6B is a cross-sectional view of the fluid flow control assembly 500 of FIG. 6A, taken at line 6B-6B of FIG. 6A. The fluid flow control assembly 500 may be configured to be positioned in-line with the gas/lens wash supply tubing 240c, 245c, 336, 338 to selectively allow a flow of fluid through the lens wash supply tubing 245c, 338. The fluid flow control assembly 500 may be positioned anywhere along a length of the lens wash supply tubing 245c between the first end thereof and the gas/lens wash connector 290. The illustrative fluid flow control assembly 500 is configured to be coupled to a coaxially arranged gas/lens wash supply tubing 240c, 245c, 336, 338. However, the fluid flow control assembly 500 may be reconfigured to be coupled to gas/lens wash supply tubing 240c, 245c, 336, 338 which extend side-by-side.

The fluid flow control assembly 500 may extend from a first end 502 to a second end 504. Generally, the first and second ends 502, 504 of the fluid flow control assembly 500 may be configured to be coupled to the gas/lens wash supply tubing 240c, 245c, 336, 338. It is contemplated that the gas/lens wash supply tubing 240c, 245c, 336, 338 may include a first segment or first length of tubing extending from the first end thereof (in fluid communication with the reservoir 270, 330) to a second end coupled to the flow control assembly 500 and a second segment or second length of tubing extending from the fluid flow control assembly 500 to the second end thereof coupled to the gas/lens wash connector 290. The fluid flow control assembly 500 may include a first body member 506 extending from a first end 508 to a second end 510 and a second body member 512 extending from a first end 514 to a second end 516.

The first end 508 of the first body member 506 may be configured to be coupled to the second body member 512 and to receive a portion of a valve housing 526 while the second end 510 of the first body member 506 may be configured to be coupled to the gas/lens wash supply tubing 240c, 245c, 336, 338. In some examples, a first end region 518 of the first body member 506 may have a first cross-sectional outer dimension and the second end region 520 of the first body member 506 may have a second cross-sectional outer dimension smaller than the first cross-sectional outer dimension. The first end region 518 of the first body member 506 may take any shape desired such, as, but not limited to, generally hemi-spherical, conical, truncated conical, cylindrical, rectangular prisms, and the like. The second end region 520 of the first body member 506 may have a generally tubular configuration sized and shaped to couple with a segment of the gas/lens wash supply tubing 240c, 245c, 336, 338.

The second end region 520 may include an outer tubular member 528 and an inner tubular member 530. The inner tubular member 530 may extend through a lumen 532 of the outer tubular member 528. The gas supply tubing 240c, 336 may be configured to be coupled to the outer tubular member 528. In some embodiments, the outer tubular member 528 may be disposed within a lumen of the gas supply tubing 240c, 336. In other embodiments, the gas supply tubing 240c, 336 may be disposed within the lumen 532 of the outer tubular member 528. The lens wash supply tubing 245c, 338 may be configured to be coupled to the inner tubular member 530. In some embodiments, the inner tubular member 530 may be disposed within a lumen of the lens wash supply tubing 245c, 338. In other embodiments, the lens wash supply tubing 245c, 338 may be disposed within an inner lumen 524 of the first body member 506.

A generally annular lumen 522 may extend from the first end 508 of the first body member 506 towards the second end 510 of the first body member 506. The generally annular lumen 522 may be in fluid communication with the lumen 532 of the outer tubular member 528 to fluidly couple the first and second segments of the gas supply tubing 240c, 336. The lumen 524 of the first body member 506 may extend from the second end 510 to the first end 508. The diameter, or cross-sectional dimension of the lumen 524 may be greater adjacent to the first end 508 than at the second end 510. For example, the lumen 524 adjacent to the first end 508 may be sized and shaped to receive a portion of a valve housing 526.

The second end 516 of the second body member 512 may be configured to be coupled to the first body member 506 and to receive a portion of the valve housing 526 while the first end 514 of the second body member 512 may be configured to be coupled to the gas/lens wash supply tubing 240c, 245c, 336, 338. In some examples, a first end region 534 of the second body member 512 may have a first cross-sectional outer dimension and the second end region 536 of the second body member 512 may have a second cross-sectional outer dimension greater than the first cross-sectional outer dimension. The first end region 534 of the second body member 512 may have a generally tubular configuration sized and shaped to couple with a segment of the gas/lens wash supply tubing 240c, 245c, 336, 338. The second end region 536 of the second body member 512 may take any shape desired such, as, but not limited to, generally hemi-spherical, conical, truncated conical, cylindrical, rectangular prisms, and the like. In some cases, the first and second body members 506, 512 may be approximate mirror images of one another, although this is not required.

The first end region 534 of the second body member 512 may include an outer tubular member 538 and an inner tubular member 540. The inner tubular member 540 may extend through a lumen 542 of the outer tubular member 538. The gas supply tubing 240c, 336 may be configured to be coupled to the outer tubular member 538. In some embodiments, the outer tubular member 538 may be disposed within a lumen of the gas supply tubing 240c, 336. In other embodiments, the gas supply tubing 240c, 336 may be disposed within the lumen 542 of the outer tubular member 538. The lens wash supply tubing 245c, 338 may be configured to be coupled to the inner tubular member 540. In some embodiments, the inner tubular member 540 may be disposed within a lumen of the lens wash supply tubing 245c, 338. In other embodiments, the lens wash supply tubing 245c, 338 may be disposed within an inner lumen 544 of the second body member 512.

A generally annular lumen 546 may extend from the second end 516 of the second body member 512 towards the first end 514 of the second body member 512. The generally annular lumen 546 may be in fluid communication with the lumen 542 of the outer tubular member 538 to fluidly couple the first and second segments of the gas supply tubing 240c, 336. For example, the generally annular lumen 546 may be fluidly coupled to the generally annular lumen 522 of the first body member 506. The inner lumen 544 of the second body member 512 may extend from the first end 514 to the second end 516. The diameter, or cross-sectional dimension of the lumen 544 may be greater adjacent to the second end 516 than at the first end 514. For example, the lumen 544 adjacent to the second end 516 may be sized and shaped to receive a portion of a valve housing 526.

The valve housing 526 may extend from a first end 548 to a second end 550. A first end region 552 of the valve housing 526 may be coupled or secured to the second body member 512 and a second end region 554 of the valve using 526 may be coupled or secured to the first body member 506. For example, the first end region 552 may be disposed within a portion of the lumen 544 of the second body member 512 and the second end region 554 may be disposed within a portion of the lumen 524 of the first body member 506. It is contemplated that the valve housing 526 may be coupled to the first and/or second body member 506, 512 using a variety of techniques, such as, but not limited to, threaded engagements, snap fits, friction fits, overmolding, adhesive bonding, welding, soldering, and the like. Further, the first and second body members 506, 512 may be secured or coupled to one another using a variety of techniques, such as, but not limited to, threaded engagements, snap fits, friction fits, overmolding, adhesive bonding, welding, soldering, and the like.

A valve 556 may be disposed within a lumen 558 of the valve housing 526. The lumen 558 of the valve housing 526 may be in fluid communication with the central lumen 544 of the second body member 512 and in selective fluid communication with the central lumen 524 of the first body member 506. The lumen 558 of the valve housing 526 may have a variable diameter, or cross-sectional dimension. In some cases, the diameter of the lumen 558 may increase at a transition point to create a rim or ledge 560 upon which a portion of the valve 556 rests. The valve 556 may be positioned between the ledge 560 of the valve housing 526 and a surface 562 of the first body member 506. The valve 556 may extend across a cross-section of the lumen 558 of the valve housing 526 and may include one or more openings or passages 564 to selectively allow a flow of fluid through the valve 556.

The valve 556 may be configured to prevent fluid from freely flowing into or out of the lens wash supply tubing 245c, 338 unless a minimum opening pressure is achieved. The opening pressure may be greater than the head pressure of the fluid 285, 334 within the reservoir 270, 330. It is contemplated that the minimum pressure may be achieved by the pressure generated by the flow of air/gas through the gas supply line 240c, 336 and into the interior of the reservoir 270, 330. Once the minimum pressure is achieved, the valve 556 may open and water/fluid may flow through the lens wash supply tubing 245c, 338 at a rate sufficient to clean the lens of the endoscope 100. Once the clinician releases the gas/water valve 140, the reservoir 270, 330 may return to its static pressure which may close the valve 556 and prevent the flow of fluid into and/or out of the lens wash supply tubing 245c, 338. The valve 556 may be formed from a single monolithic piece of silicone, thermoplastic elastomer (TPE), elastic, other flexible material, or the like. In some cases, the valve 556 may be formed from more than one piece. The one or more openings 564 may be a single slit, one or more slits in the shape of a cross or “x”, a snowflake slit, a single resealable hole, or the like. In other embodiments, the valve 556 may be an umbrella valve that changes configuration in response to the pressure change. It is further contemplated that other valves which open in response to an increase in pressure and close as the pressure is lowered may also be used.

The valve 556 may be configured to remain closed under the greatest head pressure by the water/fluid 285, 334 within the reservoir (e.g., when the reservoir 270, 330 is full). Performance characteristics of the valve 556 such as, but not limited to, the head pressure the valve 556 can resist, the flow rate and/or volume of fluid flow through the valve 556 when the opening pressure is reached can be adjusted by changing the size and/or shape of the valve 556 and/or the opening 564, material selection of the valve 556, material properties of the valve 556, the design of the first body member 506 and/or valve housing 526.

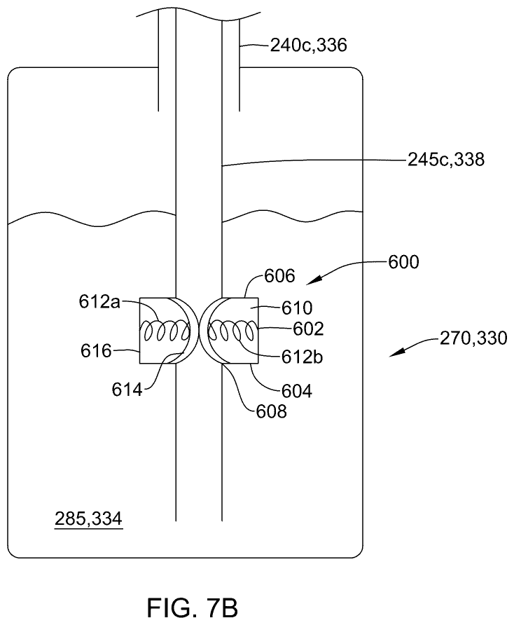

FIG. 7A depicts a schematic side view of an illustrative fluid reservoir 270, 330 having another illustrative fluid flow control assembly 600 in a first or open configuration and FIG. 7B depicts schematic side view of an illustrative fluid reservoir 270, 330 having the fluid flow control assembly 600 in a second or closed configuration. Generally, the fluid flow control assembly 600 may be a spring-loaded valve configured to selectively apply a biasing force to an exterior of the lens wash supply tubing 245c, 338 to selectively pinch the lens was supply tubing 245c, 338 closed. The fluid flow control assembly 600 may include a valve housing 602 extending from a first end 604 to a second end 606. A lumen 608 may extend through the valve housing 602 from the first end 604 to the second end 606. The lumen 608 may be sized and shaped to receive the lens wash supply tubing 245, 338 therethrough. For example, the fluid flow control assembly 600 may be slid over an exterior of the lens wash supply tubing 245, 338 such that the fluid flow control assembly 600 surround the lens wash supply tubing 245, 338 and within the reservoir 270, 330.

The valve housing 602 may include a cavity 610 therein configured to receive one or more biasing members 612a, 612b. The biasing members 612a, 612b may extend between a flexible inner membrane or wall 614 and a more rigid outer wall 616. The flexible inner wall 614 may form the portion of the valve housing 602 in contact with the exterior of the lens wash supply tubing 245, 338. Generally, the one or more biasing members 612a, 612b may be configured to exert a biasing force on the flexible inner wall 614 to move the wall of the lens wash supply tubing 245, 338 radially inward to pinch the lens wash supply tubing 245, 338 closed. The one or more biasing members 612a, 612b may be a spring or other mechanism configured to exert a biasing force on the flexible inner wall 614. The flexible inner wall 614 may be formed from silicone, thermoplastic elastomer (TPE), elastic, other flexible material configured to allow the inner wall 614 to move between a closed configuration (FIG. 7B) which pinches the wall of the lens wash supply tubing 245, 338 together to prevent a flow of fluid therethrough and an open configuration which allows fluid to flow through the lens wash supply tubing 245, 338.

The fluid flow control assembly 600 may be configured to prevent fluid from freely flowing into or out of the lens wash supply tubing 245c, 338 unless a minimum opening pressure is achieved. The opening pressure may be greater than the head pressure of the fluid 285, 334 within the reservoir 270, 330. It is contemplated that the minimum pressure may be achieved by the pressure generated by the flow of air/gas through the gas supply line 240c, 336 and into the interior of the reservoir 270, 330. Once the minimum pressure is achieved, the pressure may overcome the biasing force of the one or more biasing mechanisms 612a, 612b to allow the wall of the lens wash supply tubing 245c, 338 to radially expand (e.g., open) and water/fluid may flow through the lens wash supply tubing 245c, 338 at a rate sufficient to clean the lens of the endoscope 100. Once the clinician releases the gas/water valve 140, the reservoir 270, 330 may return to its static pressure which may allow the biasing members 612a, 612b to once again pinch the lens wash supply tubing 245c, 338 closed to prevent the flow of fluid into and/or out of the lens wash supply tubing 245c, 338.

The fluid flow control assembly 600 may be configured to remain closed under the greatest head pressure by the water/fluid 285, 334 within the reservoir (e.g., when the reservoir 270, 330 is full). Performance characteristics of the fluid flow control assembly 600 such as, but not limited to, the head pressure the biasing mechanisms 612a, 612b can resist, the flow rate and/or volume of fluid flow through the fluid flow control assembly 600 when the opening pressure is reached can be adjusted by changing the size and/or stiffness of the one or more biasing mechanism 612a, 612b, material selection of the flexible inner wall 614, and the like.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A container and tube set arranged and configured to couple to an endoscope for use in an endoscopic procedure, the container and tube set comprising:
a container configured to contain a fluid;
a water supply tube including a first end, a second end, and a first lumen extending therethrough, wherein the first lumen is in selective fluid communication with the container and the second end of the water supply tube is positioned external to the container;
a gas supply tube including a first end, a second end, and a second lumen extending therethrough, wherein the second lumen is in operative fluid communication with the first container and the second end of the first gas supply tube is positioned external to the first container; and
a fluid flow control assembly positioned in-line with the water supply tube, the fluid flow control assembly configured to selectively control a flow of fluid through the water supply tube;
wherein the fluid flow control assembly is configured to allow a flow of fluid in response to an opening pressure.

2. The container and tube set of claim 1, wherein the opening pressure is greater than a head pressure of the fluid within the container.

3. The container and tube set of claim 1, wherein the fluid flow control assembly is coupled to the first end of the water supply tube.

4. The container and tube set of claim 1, wherein the fluid flow control assembly is coupled adjacent to the second end of the water supply tube.

5. The container and tube set of claim 1, wherein the fluid flow control assembly is positioned between the first end and the second end of the water supply tube.

6. The container and tube set of claim 1, wherein the fluid flow control assembly is disposed over the water supply tube.

7. The container and tube set of claim 1, wherein the fluid flow control assembly comprises:
a first body member;
a valve housing;
a lumen extending through the first body member and the valve housing; and
a valve extending across the lumen and disposed between the body portion and the valve housing.

8. The container and tube set of claim 7, wherein the valve comprises one or more openings extending therethrough.

9. The container and tube set of claim 7, wherein the valve comprises a flexible slit valve.

10. The container and tube set of claim 7, wherein the fluid flow control assembly further comprises a second body member.

11. The container and tube set of claim 10, wherein the valve housing is disposed between the first body member and the second body member.

12. The container and tube set of claim 10, wherein a first end of the fluid flow control assembly is coupled to a first segment of the water supply tube and a first segment of the gas supply tube and a second end of the fluid flow control assembly is coupled to a second segment of the water supply tube and a second segment of the gas supply tube.

13. The container and tube set of claim 1, wherein the fluid flow control assembly comprises:
a valve housing defining a cavity; and
one or more biasing mechanisms disposed within the cavity, the one or more biasing mechanisms configured to exert a biasing force on a wall of the water supply tube to move the wall radially inward.

14. The container and tube set of claim 13, wherein the valve housing comprises a flexible inner membrane, the flexible inner membrane positioned against the water supply tube.

15. The container and tube set of claim 13, wherein the one or more biasing mechanisms comprise a spring.

16. A container and tube set arranged and configured to couple to an endoscope for use in an endoscopic procedure, the container and tube set comprising:
a container configured to contain a fluid;
a water supply tube including a first end, a second end, and a first lumen extending therethrough, wherein the first lumen is in selective fluid communication with the container and the second end of the water supply tube is positioned external to the container;
a gas supply tube including a first end, a second end, and a second lumen extending therethrough, wherein the second lumen is in operative fluid communication with the first container and the second end of the first gas supply tube is positioned external to the first container; and
a fluid flow control assembly positioned in-line with the water supply tube, the fluid flow control assembly comprising:
a body member having a first end region having a first outer dimension and a second end region having a second outer dimension, the second outer dimension less than the first outer dimension;
a valve housing secured to the second end region of the body member;
a lumen extending through the first body member and the valve housing; and
a valve extending across the lumen and disposed between the body portion and the valve housing;
wherein the fluid flow control assembly is configured to allow a flow of fluid in response to an opening pressure.

17. The container and tube set of claim 16, wherein the valve comprises one or more openings extending therethrough.

18. The container and tube set of claim 17, wherein the one or more openings comprise a single slit, one or more slits in the shape of a cross or “x”, a snowflake slit, or a resealable hole.

19. A container and tube set arranged and configured to couple to an endoscope for use in an endoscopic procedure, the container and tube set comprising:
a container configured to contain a fluid;
a water supply tube including a first end, a second end, and a first lumen extending therethrough, wherein the first lumen is in selective fluid communication with the container and the second end of the water supply tube is positioned external to the container;
a gas supply tube including a first end, a second end, and a second lumen extending therethrough, wherein the second lumen is in operative fluid communication with the first container and the second end of the first gas supply tube is positioned external to the first container; and
a fluid flow control assembly positioned in-line with the water supply tube, the fluid flow control assembly comprising:
a first body member;
a second body member;
a valve housing secured between the first body member and the second body member;
a third lumen extending from a first end to a second end of the fluid flow control assembly, the third lumen in selective fluid communication with the first lumen of the water supply tube;
a fourth lumen extending from the first end to the second end of the fluid flow control assembly, the fourth lumen in fluid communication with the second lumen of the gas supply tube; and
a valve extending across the third lumen and disposed between the first body portion and the valve housing;
wherein the fluid flow control assembly is configured to allow a flow of fluid in response to an opening pressure.

20. The container and tube set of claim 19, wherein the fluid flow control assembly is configured to be positioned between the first ends and the second ends of the water supply tube and the gas supply tube.
